(19) United States
(12) Patent Application Publication
(10) Pub. No.: US 2009/0156974 A1
(43) Pub. Date: Jun. 18, 2009

(54) Absorbent wound care device

(76) Inventors: Jens Hog Truelsen, Helsingor (DK); Brian Nielsen, Gorlose (DK)

Correspondence Address: JACOBSON HOLMAN PLLC, 400 SEVENTH STREET N.W., SUITE 600, WASHINGTON, DC, 20004, US

(21) Appl. No.: 11/991,082

(22) PCT Filed: Aug. 31, 2006
(86) PCT No.: PCT/DK2006/000469

§ 371 Date: Feb. 27, 2008

(30) Foreign Application Priority Data
Aug. 31, 2005 (DK) PA 2005 01206
Sep. 22, 2005 (DK) PA 2005 01326

(51) Int. Cl. A61F 13/00

(52) U.S. Cl. 602/45; 602/47; 602/54

(57) ABSTRACT

An absorbent wound care device comprising a wound contacting layer comprising an absorbent non-woven bi-component staple fibre material, said staple fibre material comprising fibres having at least one outside portion of highly hydrophilic material and least one inner core of a low-absorbing material, wherein the staple fibre material comprising fibres having at least one outside portion of highly hydrophilic material and least one inner core of a low-absorbing material constitutes above 50% of the fibrous material and wherein the absorbent material consists of mechanically intertwined fibres enables the use of an absorbent material comprising only fibres having at least one outside portion of highly hydrophilic material without having to rely on the presence of wicking fibres, which material is capable of absorbing liquid parts and slough from wound exudates without giving rise to gel blocking and also reduces of the risk of maceration due to wicking of moisture laterally in the plane of the wound to the neighbouring skin.

### BACKGROUND OF THE INVENTION

1. Field of the Invention

The invention relates to absorbent wound care devices, especially wound dressings comprising absorbent synthetic fibres and being suitable for handling exuding wounds.

Exudates from chronic wounds may comprise two major components, a liquid part and typically a varying amount of a more solid/highly viscous part called slough. The slough has a high content of proteins and other high-molecular compounds and may often cause problems with regard to the absorption capacity of the dressing as the slough may deposit on the surface of an absorbent element thereby causing blocking of the access to the absorbing element and preventing the uptake of further liquid part of the exudates. This is a highly undesired situation where the absorbency of a dressing is inhibited long before the absorption capacity of a dressing has been reached.

It is desirable that wound dressings for treatment of exuding wounds are capable of absorbing exudates in such manner that both the liquid part of the exudates and the slough is able to pass the surface of the dressing freely and penetrate into the dressing thereby assuring continuous managing of the body exudates during the wound heeling process. It is advantageous for the wound healing that the slough is removed from the wound bed.

Wound dressings provided with layers for absorbing body fluids are known in the art. Absorbent layers are provided for the uptake of body fluids, especially wound exudates, so as to enable the wound dressing to keep a constant moist environment over the wound site, and at the same time avoiding maceration of the skin surrounding the wound.

Much effort has been made for enhancing the rate of uptake and increasing the capacity of absorbing body fluids of a wound dressing, in particular the absorbent layer. As absorbent layers in wound dressings have been used hydrocolloids, super absorbents, foams and synthetic materials that have extensive capacity to absorb body fluids, especially wound exudates.

However, increasing the capacity and rate of fluid uptake of a wound dressing may give rise to problems, both in assuring that the body fluids do not migrate from the wound dressing and in assuring that the wound does not dry out.

Hydrocolloid dressings are some of the most efficient and broadly used dressings, being skin-friendly, absorbent and capable of creating moist wound healing conditions. However, when used on some exuding wounds, the absorption rate tends to be too low. The advantages of the hydrocolloid dressing are the ability of creating a moist wound-healing environment and acting as a barrier against contamination with bacteria.

A problem frequently arising when treating exuding wounds is maceration. Usually the absorbent part of the dressing is optimised for absorption substantially perpendicularly to the skin, so that the skin surrounding the wound is not exposed to the exudates in order to avoid maceration of this healthy, but fragile skin. However, this limits the absorption capacity of the dressing to the part of absorbent material being directly over the wound. A barrier cream/skin conditioning paste, such as zinc paste, may be used to protect the surrounding skin for neutralising the impact of a pressure sensitive adhesive and in order to avoid the maceration. However, the use of a paste will often inhibit both the adhesive tack of the dressing and limit the capability of absorbing exudates increasing the risk of leakage for products comprising pressure sensitive adhesives, as skin and the adhesive area is covered with a lipid containing layer, such as a zinc paste or silicone-products, and it is time consuming for the healthcare staff to apply.

The skin surrounding leg ulcers is often very fragile and thus easily damaged. Wound dressings that allow direct contact between pressure sensitive adhesives, such as hydrocolloid adhesives or medical acrylate based adhesives, and the fragile skin surrounding the ulcers are often not suitable for treatment of such wounds. Traumatic removal of the adhesive dressings with damage to the fragile skin is common, and suggests the use of non-adhesive products.

Highly exuding wounds are often treated with alginate based dressings, which are capable of absorbing high amounts of exudates but require additional cover dressings. Furthermore, the risk of maceration is high due to migration of moisture in the alginate dressing along the skin. Still further, Ca-alginate fibres dissolve due to a sodium-calcium ion exchange rendering the removal of the dressing in one piece difficult.

Non-woven dressings made from pectin, Ca-alginate or CMC have been used for the treatment of exuding wounds. However, these dressings have a tendency to disintegrate upon absorption due to the loss of the fibre strength. The loss of the fibre strength of the Ca-alginate fibres is due to a Ca/Na ion exchange that takes place upon absorption of the Na containing wound exudates. The alginate and CMC fibres lose the strength upon absorption due to the lack of physical bonding sites in the material. Besides loss of fibre strength in alginate and alginate and CMC dressings upon absorption, the CMC dressings tend to show film formation on the edges upon drying and stick to the wound upon removal if the wound becomes less exuding or if used on only slightly exuding wounds. Still further on drying, they do not regain their fibrous form but produce brittle sheets having a tendency to disintegrate and leave residues in the wound upon removal.

The skin may be damaged to a degree that exudates trickle from the skin surrounding the wound, being a challenge to the function of most of the well-known dressings. Furthermore, the area of the lower limbs may pose special demands to the flexibility properties of the dressing due to the anatomical complexity of the area combined with the motility of the joints of the ankle and the malleolus.

Another important consideration in the treatment of leg ulcers is that the dressing must not give rise to formation of pressure sores. A non-adhesive dressing is usually combined with compression therapy. Thus, a dressing being soft and without sharp edges is preferred.

Leg ulcers are known to be highly exuding, and may give rise to increased risk of leakage and maceration, if the wound dressing used does not show a sufficient capacity for handling exudates with respect to time and amount. Hydrocolloid products will often be unsuitable for use in wound healing stages with medium to high level of exudates, especially if the skin is covered with a lipid containing products.

2. Description of the Related Art

In published UK patent application No. GB 2 377 177 is disclosed a wound dressing comprising a layer of a low adherent gel forming fabric backed by and in liquid contact with a layer of a material having a superabsorbent component. The gel forming fabric is preferably haemostatic and made from calcium or sodium alginate fibres. It may be woven or knitted but is preferably a non woven, needlepunched fabric of basis weight 25-200 g/m². The alginate web may be medicated or contain therapeutically active metal ions. The superabsorbent component may be a powder or woven material but is preferably a needlepunched or air laid non woven fabric made of polyacrylate fibre, basis weight 50-350 g/m². In GB 2 377 177 it is stated that although alginate is gel-forming it has surprisingly been found that in the dressings disclosed therein, liquids such as blood nevertheless can pass through the gel-forming alginate layer and be absorbed by the superabsorbent layer. Furthermore, it is stated that gelling can be a relatively slow process. Thus, after absorbing liquid such as blood, the fabric of alginate fibres will eventually form a gel blocking the further access to the below super absorbent layer.

International Patent Publication No. WO 02/15816 discloses an elastic adhesive wound compression dressing for control of bleeding and for dressing bleeding wounds. The compression dressing comprises a self-adhering elastic bandage strip designed for exerting a compressive force when wrapped around a body part sufficient to hold the compression dressing in place for a period of time to provide a therapeutic effect to a wound and an absorbent pad affixed to an inner side of a terminal portion or to a terminal end of the self-adhering elastic bandage strip.

A material made of melt blown micro fibre webs may be used in the strip. The melt blown micro fibre webs may be composed of a variety of well known thermoplastic elastomers. In addition the melt blown micro fibre webs may include, staple fibres, such as rayon, polyester, nylon, cotton, LANSEAL® fibres, cellulose, or polypropylene fibres, to provide a blend of elastomeric and staple fibres. The dressing is not intended to absorb large amounts of blood, but allows hygienic isolation of the area and prevents excess bleeding by sealing the wounded region and compressing and thereby blocking ruptured blood vessels. Preferably, the absorbent material both absorbs wound exudates and protects the wound by absorbing shocks.

U.S. Pat. No. 4,650,479 discloses sorbent sheet products useful in disposable incontinent devices, diapers, surgical swaps, bed pads, sanitary napkins, and wipers. The products comprises a coherent fibrous web that includes entangled blown fibres and high absorbency, liquid absorbent staple fibres intermingled with the blown fibres and uniformly dispersed and physically held within the web, the high-absorbency, liquid absorbent staple fibres swelling upon sorption of liquid. Additionally, the web may contain other constituents such as wetting agents and liquid conductive, liquid transport fibres which wick or transport the liquid into away from the wound and into the web.

To achieve high liquid absorbency and good liquid retention under pressure, the absorbent staple fibre should have at least one outside portion of highly hydrophilic material. Examples of such highly hydrophilic fibres are those prepared by treating acrylonitrile fibres with an alkali-metal hydroxide to form a hydrophilic cross-linked polymer on the surface thereof. Also useful are fibres having an absorbent coating such as a cross-linked, saponified copolymer of methacrylic acid and ethacrylic acid or a homopolymer of acrylic acid. Particularly useful fibres are “Lanseal® F” fibres, an acrylonitrile fibre having a hydrophilic cross-linked polymer on the surface thereof, available from Japan Exian Co., Ltd., Osaka, Japan.

In U.S. Pat. No. 4,650,479 it was found that if the product comprised 50% or more absorbent fibres, the absorption of water was slower due to gel blocking, a condition where the sorbent fibres with sorbed liquid on the outer portion of the sheet form a gel which then acts as a dam inhibiting liquid from passing to the inner portions of the sheet.

Further it was found that when adding a surfactant, slower absorption was observed for low contents of absorbing fibres, and gel blocking was observed for a product comprising about 60% absorbing fibres. A product comprising only absorbing fibres showed low absorption due to gel blocking.

Soft, flexible, absorbent and coherent dressings are in general needed for providing a proper treatment of exuding wounds not giving rise to formation of pressure marks or leaving dressing residues in the wound upon removal.

In some cases, e.g. diabetic foot ulcers, soft, flexible and cuttable dressings are especially needed for difficult accessible wounds. Cavity wounds need a flexible/conformable dressing which is easy to fit in the cavity.

Commercially available non-woven materials containing Lanseal® fibres contain liquid conductive fibres (wicking fibres) in order to prevent gel blocking and to increase the overall absorption of the non-woven fabric and not limit the absorption to the area actually been wetted. However, spreading of the absorbed liquid to the dressing outside the wound area is undesirable as it may lead to maceration.

Thus, there is still a need for a non-adhesive flexible, highly absorbent wound dressing being capable of absorbing large amounts of wound exudates comprising slough without giving rise to gel blocking and/or maceration.

One object of the present invention is to provide a wound care device being capable of handling slough.

Another object of the present invention is to provide a device for treatment of highly exuding wounds.

Yet another object of the present invention is to provide a wound care device, which is less susceptible to gel blocking.

Still another object of the present invention is to provide an absorbent wound care device showing only moderate expansion, when wetted.

Still a further object of the present invention is to provide an absorbing wound care device which has a sufficient cohesion to be removed in one piece essentially without leaving remnants in a wound bed.

The present invention provides a solution to the above objects enabling the use of an absorbent material comprising only fibres having at least one outside portion of highly hydrophilic material without having to rely on the presence of wicking fibres.

### SUMMARY OF THE INVENTION

In a first aspect the invention relates to an absorbent wound care device comprising a wound contacting layer comprising an absorbent non-woven bi-component staple fibre material, said staple fibre material comprising fibres having at least one outside portion of highly hydrophilic material and least one inner core of a low-absorbing material, wherein the staple fibre material comprising fibres having at least one outside portion of highly hydrophilic material and least one inner core of a low-absorbing material constitutes above 50% of the fibrous material and wherein the absorbent material consists of mechanically intertwined fibres.

In a second aspect the invention relates to the use of a material consisting of mechanically intertwined fibres having a double layer structure with an outer layer of a super absorbent material and least one inner core of a low-absorbing material to form a wound care device.

### DETAILED DESCRIPTION OF THE PRESENT INVENTION

The present invention relates to an absorbent wound care device comprising a wound contacting layer comprising an absorbent non-woven bi-component staple fibre material, said staple fibre material comprising fibres having at least one outside portion of highly hydrophilic material and least one inner core of a low-absorbing material, wherein the staple fibre material comprising fibres having at least one outside portion of highly hydrophilic material and least one inner core of a low-absorbing material constitutes above 50% of the fibrous material and wherein the absorbent material consists of mechanically intertwined fibres.

Contrary to expectation it has been found that it is possible to use of an absorbent material comprising a proportion about 50% of fibres having at least one outside portion of highly hydrophilic material without having to rely on the presence of wicking fibres for handling highly exuding wounds without risking gel blocking. The absence of wicking fibres reduces the risk of transportation of moisture laterally in the plane of the wound to the neighbouring skin and thus the risk of maceration.

The mechanical intertwining may be made in a manner known per se and is preferably carried out by needle punching the material giving rise to a structure in which a part of the fibres are not arranged parallel to the wound contacting layer but form an angle to plane of the wound contacting layer, allowing the material to absorb fluid from the wound, thereby allowing swelling without blocking absorption of the material.

A needle punched wound care device of the invention preferably has been needled to a degree ensuring that the resulting device shows only moderate expansion when wetted and also ensuring sufficient space between the swollen fibres for exudates to pass into the material and slough to be removed from the wound bed. In one embodiment of the invention, the part of the fibres forming an angle to plane of the wound contacting layer is substantially parallel.

In one embodiment of the invention the needle punched fibre material has been needled to a density of from about 85 to about 350 punches per cm², suitably from about 85 to about 275 punches per cm². It has been found that even when using a needle punched fibre material having such high degrees of needling density no gel blocking is observed.

It has been found that an absorbing wound care device of the invention has a sufficient cohesion to be removed in one piece essentially without leaving remnants in the wound bed.

Furthermore, it has been found that the wound care device of the invention provides a higher capacity of absorbing a standard solution for measuring absorption of wound care devices than the dressings disclosed in GB 2 377 177 and also provides a higher retention under pressure.

The length of the fibres may be from 10 to 100 millimetres more preferred 20 to 80 mm and even more preferred from 25 to 75 mm and most preferred from 30 to 60 mm.

Suitable staple fibres for use in accordance with the present invention are fibres having a double layer structure with an outer layer of a super absorbent material and an inner core of a low absorbing material such as a polyolefin such as PE or PP, an acrylic polymer, a polyurethane, polystyrene, a PVA having a high degree of hydrolysis of about 99% or PVC, preferably an acrylic fibre. Such fibrous material provides a high absorption of liquid and still shows sufficient coherency to be removed without leaving remnants or sticking to the wound or skin around the wound.

The super absorbent material is preferably a cross-linked polymer enabling a high absorption of liquid without disintegration of the material. A very suitable fibre material to be used in accordance with the present invention is Lanseal® fibres in the dimensions 5.6 dtex×51 millimetres or 2.9 dtex×38 millimetres.

An absorbing wound care device of the invention suitably has a density ranging from 0.02 to 0.3 g/cm³, more preferred from 0.025 to 0.15 g/cm³, and most preferred from 0.03 to 0.1 g/cm³, in particular from 0.03 to 0.07 g/cm³.

In an embodiment of the invention the absorbing wound care device is calendered.

The thickness of an absorbing wound care device of the invention may suitably be up to 10 millimetres, more preferred from 2 to 7 millimetres. If calendered, the thickness of an absorbing wound care device of the invention may typically vary from 1 to 3 millimetres.

The absorbent material of the invention may further comprise different fibres selected from binding fibres such as low melting bi-component PET fibres melt blown fibres such as polyethylene fibres, polypropylene fibres, polyethylene terephthalate fibres, and polyamide fibres, solution blown fibres such as fibres of polymers or copolymers of vinyl acetate, vinyl chloride, and vinylidene chloride, and/or absorbing fibres of hydrophilic fibres such as acrylonitrile fibres treated with an alkali-metal hydroxide. It is preferred that additional fibres are not wicking fibres spreading moisture laterally in the plane of the wound to the neighbouring skin and thus the risk of maceration. Additional absorbing fibres may e.g. be alginate fibres, CMC fibres, polyether-polyurethane fibres, chitosan fibres, carboxymethyl-chitin fibres, or fibres of amphiphilic block copolymers such as a polystyrene-PEG-polystyrene copolymer.

The content of fibres which may be present in addition to the fibres having a double layer structure with an outer layer of a super absorbent material and an inner core of a low absorbing fibre in the materials of the invention is preferably below about 30%. The content of fibres having a double layer structure with an outer layer of a super absorbent material and an inner core of a low absorbing fibre in the materials of the invention is suitably about 75%, more preferred above about 80% end even more preferred above about 90%.

In a preferred embodiment of the invention at least 70% of fibres of the absorbing material are absorbing fibres and wherein at least 50% of fibres of the absorbing material are bi-component fibres.

A wound care device of the invention may consist of a needle punched absorbent staple fibre material wherein the fibres have at least one outside portion of highly hydrophilic material, preferably having a double layer structure with an outer layer of a super absorbent material and an inner core of a low-absorbing material such as an acrylic fibre. In this case, the device is non-adhesive and will normally be used together with a cover dressing or surgical dressing materials.

In an alternative embodiment of the invention the absorbent wound care device is a part or an element of a wound dressing. The wound dressing may be in the form of a layer of a needle punched absorbent staple fibre material wherein the fibres have at least one outside portion of highly hydrophilic material and least one inner core of a low-absorbing material, said device having one surface which is provided with a skin friendly adhesive for attachment to the skin. In one embodiment of the invention the opposite (non-skin-facing) surface of the layer is provided with a backing layer, preferably a backing layer in the form of a film.

A dressing of the invention comprising a separate absorbing element is suitably in the form of an “island dressing” in which the absorbing element is encircled by an adhesive border. The dressing may have any appropriate shape such as circular, oval, square or rectangular.

In one embodiment of the invention the dressing is in the form of a string, rope or elongated strip. The string or strip may be used as cavity filler for deep wounds and may be crumpled to snug-fit the dimensions of the wound.

A device of the invention may comprise one or more active ingredients, such as pharmaceutically or biologically active ingredients.

Examples of such ingredients may be bacteriostatic or bactericidal compounds, enzymes for cleansing of wounds, e.g. pepsin, trypsin and the like, proteinase inhibitors or metalloproteinase inhibitors or pain killing agents.

A cover dressing to be used together with a device of the invention may be a cover dressing known per se such as an Opsite® Dressing, a highly permeable film from Smith & Nephew, or a Mesorb® Dressing, a cellulose pulp dressing from Mölnlycke, or other traditional cellulose pulp containing dressing.

Skin-friendly adhesives may suitably be a medical grade barrier adhesive known in the art such as the formulations being disclosed, for example in U.S. Pat. Nos. 4,367,732, 6,171,594, or 6,303,700, or in WO Application No. 00/54820.

A backing layer may be of any suitable layer known per se for use as backing layer of wound dressings e.g. a foam layer, or a non-woven layer or a polyurethane, polyethylene, polyester or polyamide film, preferably a polyurethane film.

A wound care device of the invention having a skin-contacting surface being provided with a skin-friendly adhesive is optionally covered in part or fully by one or more release liners or cover films to be removed before or during application.

A protective cover or release liner may for instance be siliconized paper. It does not need to have the same contour as the device, and a number of devices may be attached to a larger sheet of protective cover. The protective cover is not present during the use of the device of the invention and is therefore not an essential part of the invention.

Furthermore, the device of the invention may comprise one or more “non touch” grip (s) known per se for applying the device to the skin without touching the adhesive layer. Such a non-touch grip is not present after application of the dressing. For larger devices it is suitable to have 2 or 3 or even 4 “non-touch” grips.

In a second aspect the invention relates to the use of a material consisting of mechanically intertwined fibres having a double layer structure with an outer layer of a super absorbent material and an inner core of an acrylic fibre to form a material for a wound care device.

In a third aspect the invention relates to the use of a material consisting of mechanically intertwined fibres of an absorbent non-woven staple fibre material wherein the fibres have at least one outside portion of highly hydrophilic material and least one inner core of a low-absorbing material to form a wound care device.

Such wound care devices are capable of taking up exudates from highly exuding wounds without gel blocking and also reduces of the risk of maceration due to wicking of moisture laterally in the plane of the wound to the neighbouring skin.

For the purpose of the present invention the expression “highly hydrophilic material” is used to designate a material which is capable of absorbing at least 10 grams of water per gram hydrophilic material. Preferably the absorbing capacity is at least 15 grams of water per gram hydrophilic material and more preferred at least 20 grams of water per gram hydrophilic material.

For the purpose of the present invention the expression “low-absorbing material” is used to designate a material which has absorption below 5 grams of water per gram material, more preferred below 1 grams of water per gram hydrophilic material and preferably about 0 grams of water per gram hydrophilic material.

### Materials and Methods

Lanseal® F fibres (2.9 dtex, 38 mm and 5.6 dtex, 51 mm) are obtainable from Toyobo co., Ltd. Advanced Polymer Department, 2-8, Dojima Hama 2-Chome, Kita-Ku, Osaka, 530-8230, JAPAN.

A roller-carding machine having a receive drum.

A Vulkan type stitching machine was used.

Calender, a laboratory calender having a nip of 1.3 millimetres between the rollers was used.

A complete Falubaz flat card line with feeder. The line was specially designed to process short staple of cotton type fibres.

Measurements of thickness of materials needle-punched non-woven materials were performed according to the standard method according to EN 29073-T2.

Absorption of Solution A (prepared according to EN13726-1) of samples was measured according Danish/European standard DS/EN 13726-1 and during application of a pressure of 40 mmHg (5332.9 Pa).

Absorption under pressure was measured by placing an oblate of the product with a predetermined diameter between a porous glass-filter placed in a Petri dish and a POM (polyoxymethylene) plate. A load equal to 40 mmHg was placed on top of the POM plate. The porous glass-filter was saturated with Solution A and the product was left to absorb for 24 h at room temperature. The residual Solution A was removed after 24 h and the load was removed. The absorbency was calculated according to following equations:

$$g \text{/} g \text{:} Absorption = \frac{{w}_{24 h} - {w}_{0 h}}{{w}_{0 h}}$$

$$g \text{/} {cm}^{2} \text{:} Absorption = \frac{{w}_{24 h} - {w}_{0 h}}{{A}_{Oblate}}$$

### Where:

w₀ₕ is the initial weight of the oblate.
w₂₄ₕ is the weight of the oblate after 24 h absorption.
A_{Oblate} is the area of the oblate.

### Examples 1-2

### Preparation of Needle Punched Non-Woven Materials According to the Invention from Lanseal® F Fibres

Using a modernised laboratory roller carding machine made by Joseph Co. having a working width of 0.6 metres fleeces were prepared from each 0.5 kilograms of 2.9 dtex/38 mm and 5.6 dtex/51 mm Lanseal® F fibres, respectively. The fleece was collected on a drum, which served as the take-up unit, by superposing/multiplying small fleeces from the collector.

It was found empirically that 24-folded single pieces of fleece would be a suitable intermediate product for preparing a non-woven having a basis weight GSM of 150 g/m². From each 0.5 kg portion of fibres five fleece samples were prepared each having a weight of about 100 grams and a length of 1.1 metre. The fleece samples were then needled using a Vulkan type stitching machine having a working width of 1 m. The fleece was needled from both sides with 56 needle punchings on each side (112 in all). The specific weight, the thickness, the density and the absorption according to DS/EN13726-1 and under pressure of the resulting products appear from the below Table 1

TABLE 1

| Example | | | 1 | 2 |
|---|---|---|---|---|
| Type of Lanseal F fibres | | | 2.9 dtex/38 | 5.6 dtex/51 |
| | | | mm | mm |
| Specific weight of the needle | | | 160 | 145 |
| punched non-woven, g/m² | | | | |
| Thickness, mm | | | 4 | 4 |
| Density, g/cm³ | | | 0.040 | 0.036 |
| Needle density (# per cm²) | | | 112 | 112 |
| Absorption | DS/EN | g/100 cm² | 30 | 43 |
| of Solution | 13726-1 | g/g | 27 | 23 |
| A | Under | g/100 cm² | 24 | 29 |
| | pressure | g/g | 21 | 14 |

### Examples 3-4

### Preparation of a Needle Punched Non-Woven Materials of the Invention from Lanseal® F Fibres

In the manner described in Example 1 fleece materials were made from 160 grams and 220 grams, respectively, 5.6 dtex/51 mm Lanseal® F fibres using a roller carding machine, and the resulting materials were then needled two times using a Vulkan type stitching machine. The specific weights, the thicknesses, the densities and the absorption according to DS/EN13726-1 and under pressure of the resulting products appear from the below Table 2.

TABLE 2

| | | |
|---|---|---|
| Example | 3 | 4 |

| Specific weight of the needle | | | 140 | 190 |
|---|---|---|---|---|
| punched non-woven, g/m² | | | | |
| Thickness, mm | | | 2.7 | 3.15 |
| Density, g/cm³ | | | 0.052 | 0.06 |
| Needle density (# per cm²) | | | 178 | 178 |
| Absorption | DS/EN | g/100 cm² | 30 | 43 |
| of Solution | 13726-1 | g/g | 27 | 23 |
| A | Under | g/100 cm² | 24 | 29 |
| | pressure | g/g | 21 | 14 |

### Example 5

A part of the material from Example 4 was needled a third time on the Vulkan type stitching machine with further 56 needle punchings. The specific weight, the thickness, the density and the absorption according to DS/EN13726-1 and under pressure of the resulting product appears from the below Table 3.

TABLE 3

| Example | | | 5 |
|---|---|---|---|
| Specific weight of the needle | | | 145 |
| punched non-woven, g/m² | | | |
| Thickness, mm | | | 2.92 |
| Density, g/cm³ | | | 0.05 |
| Needle density (# per cm²) | | | 267 |
| Absorption | DS/EN | g/100 cm² | 31 |
| of Solution | 13726-1 | g/g | 23 |
| A | Under | g/100 cm² | 25 |
| | pressure | g/g | 17 |

### Examples 6-7

Fleece materials were formed from 1000 grams 5.6 dtex 51 mm Lanseal® F fibres using a complete Falubaz flat card line with feeder. The final fleece was formed from 26 single fleeces. Such fleece was needled once on a Vulkan type stitching machine as described in Example 1. A part of the needled fleece was folded in two layers (Example 6) and another part was folded in four layers (Example 7). In both Examples the resulting products were needled twice using the Vulkan type stitching machine. The specific weights, the thicknesses, the densities and the absorption according to DS/EN13726-1 and under pressure of the resulting products appear from the below Table 4.

TABLE 4

| | | |
|---|---|---|
| Example | 6 | 7 |

| Specific weight of the needle | | | 220 | 445 |
|---|---|---|---|---|
| punched non-woven, g/m² | | | | |
| Thickness, mm | | | 5.3 | 10 |
| Density, g/cm³ | | | 0.041 | 0.045 |
| Needle density (# per cm²) | | | 267 | 267 |
| Absorption | DS/EN | g/100 cm² | 59 | 116 |
| of Solution | 13726-1 | g/g | 28 | 24 |
| A | Under | g/100 cm² | 31 | 51 |
| | pressure | g/g | 16 | 12 |

### Example 8

A sample of Approximately 1 m² of the material produced in Example 6 was calendered at 160° C. using a calender. The specific weight, the thickness, the density and the absorption according to DS/EN13726-1 and under pressure of the resulting product appears from the below Table 5.

TABLE 5

| Example | | | 8 |
|---|---|---|---|
| Specific weight of the needle | | | 207 |
| punched non-woven, g/m² | | | |
| Thickness, mm | | | 1.9 |
| Density, g/cm³ | | | 0.11 |
| Needle density (# per cm²) | | | 267 |
| Absorption | DS/EN | g/100 cm² | 47 |
| of Solution | 13726-1 | g/g | 22 |
| A | Under | g/100 cm² | 29 |
| | pressure | g/g | 12 |

### Examples 9-13

A fleece material was formed from each 1300 grams 2.9 dtex/38 mm Lanseal® F fibres using a flat card line. The final fleeces were formed from 26 single fleeces. Such a fleece material was needled once on a Vulkan type stitching machine as described in Example 1 (Example 9). A part of the needled fleece material was needled for a second time (Example 10) and a part thereof for a third time (Example 11) using the Vulkan type stitching machine. A sample of the needled fleece was folded to form two layers (Example 12) and another sample was folded to form four layers (Example 13) and needled once using the Vulkan type stitching machine. The specific weights, the thicknesses, the densities and the absorption according to DS/EN13726-1 and under pressure of the resulting products appear from the below Table 6.

TABLE 6

| Example | | | 9 | 10 | 11 |
|---|---|---|---|---|---|
| Specific weight of the needle | | | 144 | 120 | 115 |
| punched non-woven, g/m² | | | | | |
| Thickness, mm | | | 3.2 | 2.7 | 2.6 |
| Density, g/cm³ | | | 0.045 | 0.044 | 0.044 |
| Needle density (# per cm²) | | | 89 | 178 | 267 |
| Absorption | DS/EN | g/100 cm² | 40 | 38 | 27 |
| of Solution | 13726-1 | g/g | 25 | 22 | 25 |
| A | Under | g/100 cm² | 26 | 21 | 16 |
| | pressure | g/g | 17 | 18 | 16 |

| | | |
|---|---|---|
| Example | 12 | 13 |

| Specific weight of the needle | | | 180 | 400 |
|---|---|---|---|---|
| punched non-woven, g/m² | | | | |
| Thickness, mm | | | 3.0 | 6.1 |
| Density, g/cm³ | | | 0.060 | 0.066 |
| Needle density (# per cm²) | | | 267 | 267 |
| Absorption | DS/EN | g/100 cm² | 38 | 68 |
| of Solution | 13726-1 | g/g | 23 | 16 |
| A | Under | g/100 cm² | 19 | 43 |
| | pressure | g/g | 12 | 9 |

### Example 14

A sample of Approximately 1 m² of the material produced in Example 10 was calendered at 160° C. using a calender. The specific weight, the thickness, the density and the absorption according to DS/EN13726-1 and under pressure of the resulting product appears from the below Table 7

TABLE 7

| Example | | | 14 |
|---|---|---|---|
| Specific weight of the needle | | | 86 |
| punched non-woven, g/m² | | | |
| Thickness, mm | | | 1.2 |
| Density, g/cm³ | | | 0.072 |
| Needle density (# per cm²) | | | 267 |
| Absorption | DS/EN | g/100 cm² | 19 |
| of Solution | 13726-1 | g/g | 24 |
| A | Under | g/100 cm² | 13 |
| | pressure | g/g | 19 |

From the above Examples it appears that the absorption of Solution A as measured in grams solution per gram material both when measured according to DS/EN13726-1 and under pressure shows little or no dependency on the density indicating that no gel blocking occurs.

1. An absorbent wound care device comprising a wound contacting layer comprising an absorbent non-woven bi-component staple fibre material, said staple fibre material comprising fibres having at least one outside portion of highly hydrophilic material and least one inner core of a low-absorbing material, wherein the staple fibre material comprising fibres having at least one outside portion of highly hydrophilic material and least one inner core of a low-absorbing material constitutes above 50% of the fibrous material and wherein the absorbent material consists of mechanically intertwined fibres.

2. A device according to claim 1 wherein the mechanically intertwined fibres are needle-punched fibres.

3. A device according to claim 1 wherein the staple fibres have a double layer structure with an outer layer of a super absorbent material and an inner core of an acrylic fibre.

4. A device according to claim 3 wherein the super absorbent material is a cross-linked polymer.

5. A device according to claim 1 wherein the absorbent staple fibre material consists of fibres having a double layer structure with an outer layer of a super absorbent material and an inner core of an acrylic fibre.

6. A device according to claim 1 wherein a part of the fibres form an angle to the plane of the wound contacting layer and are substantially parallel.

7. A device according to claim 1 wherein the fibres are needled to a degree ensuring that the resulting device shows only moderate expansion when wetted and also ensuring sufficient space between the swollen fibres for exudates to pass into the material and slough to be removed from the wound bed.

8. A device according to claim 1, said device having a density ranging from 0.02 to 0.3 g/cm³.

9. A device according to claim 1 wherein at least 70% of fibres of the absorbing material are absorbing fibres and wherein at least 50% of fibres of the absorbing material are bi-component fibres.

10. A wound care device comprising an absorbing element comprising an absorbing material according to claim 1, said device comprising a skin-contacting surface provided with a skin friendly adhesive for attachment to the skin.

11. A wound care device according to claim 10 in which the absorbent element is in the form of an “island dressing” in which the absorbing element is encircled by an adhesive border.

12. A wound care device according to claim 10 having a non-skin-facing provided with a backing layer.

13. A wound care device according to claim 1, wherein the needle punched fibre material has been needled to a density of from about 85 to about punches per cm².

14. The use of a material consisting of mechanically intertwined fibres having a double layer structure with an outer layer of a super absorbent material and least one inner core of a low-absorbing material to form a wound care device.

15. The use of a material consisting of mechanically intertwined fibres of an absorbent non-woven staple fibre material wherein the fibres have at least one outside portion of highly hydrophilic material and least one inner core of a low-absorbing material to form a wound care device.
